(19) United States
(12) Patent Application Publication
(10) Pub. No.: US 2022/0226522 A1
(43) Pub. Date: Jul. 21, 2022

(54) ULTRAVIOLET LIGHT DELIVERY APPARATUS

(71) Applicant: Lions Eye Institute for Transplant and Research, Inc., Tampa, FL (US)

(72) Inventors: Arthur C. Kurz, Safety Harbor, FL (US); Eric Abdullayev, Tarpon Springs, FL (US)

(21) Appl. No.: 17/581,074

(22) Filed: Jan. 21, 2022

Related U.S. Application Data
(60) Provisional application No. 63/140,029, filed on Jan. 21, 2021.

(51) Int. Cl. A61L 2/10

(52) U.S. Cl. CPC A61L 2/10; A61L 2202/14; A61L 2202/11

(57) ABSTRACT

A portable, handheld ultraviolet light delivery apparatus for sanitization target surfaces is configured to deliver ultraviolet light at the lower end of the UVC band (i.e., having shorter wavelengths) with the majority of the ultraviolet light having a wavelength of 222 nm. The ultraviolet light delivery apparatus includes a light source positioner secured to the front side of the case where the light source positioner is configured to maintain a constant distance between the ultraviolet light source and a target surface to be sanitized. The ultraviolet light delivery apparatus further includes a timer relay that removes power from the ultraviolet light after a specified exposure duration has elapsed so as to control the dose of ultraviolet light delivered to the target surface.

### CROSS REFERENCE TO RELATED APPLICATION

This application claims priority from U.S. provisional application No. 63/140,029 filed Jan. 21, 2021, the entirety of which is incorporated herein by reference.

### TECHNICAL FIELD AND BACKGROUND

The present invention relates generally to ultraviolet (“UV”) light devices, and more particularly, to a portable ultraviolet light delivery apparatus for sanitizing target surfaces, including organic tissue.

Ultraviolet radiation has a known germicidal effect. But conventional ultraviolet light delivery devices suffer from disadvantages with respect to safety and efficacy. Conventional ultraviolet light sources emit radiation at wavelengths that are both carcinogenic and cataractogenic, and ultraviolet light delivery devices may not effectively target the surface to be sanitized allowing radiation to propagate to areas surrounding the target surface. This undesired radiation propagation in turn results in unnecessary radiation exposure that damages cells and surrounding organic tissue.

The problems with conventional ultraviolet light delivery devices are exacerbated when users position an ultraviolet light delivery device too far from the target surface thereby causing further undesired radiation propagation while also reducing the intended germicidal effect owing to reduced radiation intensity over the target surface area. Device users may also utilize ultraviolet light exposure durations that are too long resulting in further unnecessary exposure, or in other cases, exposure durations that are too short to effectively sanitize the target surface. Simply put, the variability in user operation of an ultraviolet light delivery device can have adverse consequences with respect to safety and germicidal effectiveness.

The radiation wavelength, distance to a target surface, and exposure duration are all important factors that influence the safety and efficacy of an ultraviolet light delivery device being employed for sanitation purposes. Given the drawbacks of conventional ultraviolet light delivery devices and variations in user operation, it would be advantageous to provide an ultraviolet light delivery apparatus that is capable of delivering a measured dose that is consistent, targeted, and effective doses of radiation.

It is, therefore, an object of the present invention to provide an ultraviolet light delivery apparatus that is configured to deliver carefully controlled wavelengths of ultraviolet light that reduce harmful effects on organic tissue while still providing proper sanitization. The present ultraviolet delivery apparatus mitigates variability in user operation to deliver safe, effective, and consistent dosages of ultraviolet radiation through control of exposure times and exposure distances while also being portable, handheld, and convenient to use.

### SUMMARY

Disclosed is an ultraviolet light delivery apparatus that includes a case with a front side and a rear side that attach together to enclose components of the delivery apparatus. The front and rear side may form a handle portion that allows the apparatus to be easily transported and held in a stable position during operation of the apparatus—i.e., while ultraviolet light is emitted. An ultraviolet light source is secured to the case and has a first emission surface that emits ultraviolet light outward from the front side of the case in a direction along a first axis that extends between the front side and the rear side of the case. A light source positioner is secured to the case at a proximal end of the positioner. The light source positioner also has a distal end that is located a length, or operational distance, from the proximal end. A timer relay is secured to the case and is placed in signal communication with a power supply. The timer relay is configured to remove power to the ultraviolet light source after a specified exposure duration time period has elapsed.

Suitable ultraviolet lights sources include excimer lamps that emit ultraviolet light in the UVC range having wavelengths from 200 nanometers to 280 nanometers. More particularly, the excimer lamp can be configured so that ninety percent of the emitted ultraviolet light is at a wavelength of 222 nanometers. This can be accomplished in part by selection of the appropriate lamp as well as through the use of an ultraviolet light filter disposed proximal to the first emission surface of the ultraviolet light source.

The excimer lamp can emit light from multiple surfaces. That is, the ultraviolet light source may include a second emission surface that emits ultraviolet light in a direction along the first axis toward the rear side of the case. In that instance, the ultraviolet light delivery apparatus further includes a reflective backing secured within the case and positioned between the ultraviolet light source second emission surface and the rear side of the case. The reflective backing directs ultraviolet light emitted from the second emission surface back toward the front side of the case and toward a target surface to be sterilized.

In another aspect of the invention, the light source positioner distal end can be formed with a planar stabilizing surface that frictionally engages a target surface transverse to the first axis where the target surface receives ultraviolet light emitted by the first emission surface of the light source. The stabilizing surface holds the ultraviolet light delivery apparatus in a stable position that is a consistent operational distance from the target surface to ensure a more uniform distribution and dosing of ultraviolet light during an exposure duration period. As an example, the operational distance can be four centimeters to seven centimeters.

In a further aspect of the invention, the light source positioner inner surface is at least partially covered by an ultraviolet light reflective material. The reflective material prevents unintended dispersive emissions of ultraviolet light and helps direct the ultraviolet light toward a target surface to be sanitized. The light source positioner inner surface can alternatively be partially or entirely covered by a radiant shielding material.

In one embodiment, the light source positioner is releasably secured to the front exterior surface by a hinge. In this manner, the light source positioner can move between a closed position that at least partially covers the ultraviolet light source and an open position that allows ultraviolet light to pass. The light source positioner may be configured as two doors or covers that are secured to the case and that open and close to cover and protect the ultraviolet light source.

The ultraviolet light delivery apparatus may further include a set point input control, such as buttons or a touch screen, that interfaces with the timer relay to increase or decrease the specified exposure duration time period. The apparatus can be configured to track and store a total usage duration that represents the total amount of time an ultraviolet light source has been in operation. This allows the ultraviolet light source to be changed to prevent degradation of the emissions over time that might negatively impact the sterilization efficacy of the apparatus. In that case, the ultraviolet light delivery apparatus includes a digital processor (i.e., a CPU) that is in signal communication with the timer relay. The processor receives data from the timer relay that indicates how long the ultraviolet light has been in operation emitting ultraviolet light. The digital processor is configured to use the data received from the timer relay to determine a total usage duration for the ultraviolet light source. The apparatus also include a non-transitory electronic data storage device configured to store the total usage duration. The data storage device can be a solid state or optical storage drive.

### BRIEF DESCRIPTION OF THE FIGURES

Features, aspects, and advantages of the present invention are better understood when the following detailed description of the invention is read with reference to the accompanying figures, in which:

FIG. 1 is an exploded view of an ultraviolet light delivery apparatus according to one embodiment.

FIG. 2 is a cutaway, side view of an ultraviolet light delivery apparatus according to one embodiment.

FIG. 3A is a top, front, perspective view of an ultraviolet light delivery apparatus according to one embodiment.

FIG. 3B is a top, front, perspective view of an ultraviolet light delivery apparatus according to one embodiment.

FIG. 4 is a perspective view of an ultraviolet light delivery apparatus according to one embodiment.

FIG. 5A is front view of an ultraviolet light delivery apparatus according to one embodiment.

FIG. 5B is a cutaway, side view of an ultraviolet light delivery apparatus according to one embodiment.

FIG. 6 is a top, perspective view of an ultraviolet light delivery apparatus according to one embodiment.

FIG. 7 is an interior view of a disassembled ultraviolet light delivery apparatus according to one embodiment.

FIG. 8 illustrates experimental results for Candida Albicans growth inhibition following ultraviolet radiation exposure.

FIG. 9 illustrates experimental results for methicillin-resistant staphylococcus aureus (“MRSA”) growth inhibition following ultraviolet radiation exposure.

FIG. 10A illustrates experimental results for Candida Tropicana and MRSA growth inhibition following ultraviolet radiation exposure.

FIG. 10B illustrates experimental results for Candida Albicans and MRSA growth inhibition following ultraviolet radiation exposure.

### DETAILED DESCRIPTION

The present invention will now be described more fully hereinafter with reference to the accompanying pictures in which exemplary embodiments of the invention are shown. However, the invention may be embodied in many different forms and should not be construed as limited to the representative embodiments set forth herein. The exemplary embodiments are provided so that this disclosure will be both thorough and complete and will fully convey the scope of the invention and enable one of ordinary skill in the art to make, use, and practice the invention.

Relative terms such as lower or bottom; upper or top; upward, outward, or downward; forward or backward; and vertical or horizontal may be used herein to describe one element's relationship to another element illustrated in the figures. It will be understood that relative terms are intended to encompass different orientations in addition to the orientation depicted in the drawings. By way of example, if a component in the drawings is turned over, elements described as being on the “bottom” of the other elements would then be oriented on “top” of the other elements. Relative terminology, such as “substantially” or “about,” describe the specified materials, steps, parameters, or ranges as well as those that do not materially affect the basic and novel characteristics of the claimed inventions as whole (as would be appreciated by one of ordinary skill in the art).

The terms “sanitizing,” “disinfecting,” “germicidal,” “antimicrobial,” and variations of these terms, are used interchangeably in this application to refer to the reduction or elimination of bacteria, viruses, fungi, microbes, germs, and other harmful contaminants from a target surface. The terms “microorganism” or “microbe” are used in this application to refer generally to single-cell or multi-cell contaminants, such as bacteria, fungi, archaea, protists, viruses, and the like that are reduced or eliminated through exposure to ultraviolet radiation.

Disclosed is a portable, handheld ultraviolet light delivery apparatus for sanitization target surfaces that is configured to deliver specified wavelength(s) of ultraviolet light for a specified period of time and at a consistent distance from the target surface. The ultraviolet light wavelength, exposure duration, and distance to a target surface are all factors that impact the safety and efficacy of a sanitization device, and the embodiments of the ultraviolet light delivery apparatus disclosed in this application provide the ability to accurately and precisely control these factors. The apparatus finds particular application in disinfecting human or animal tissue, including ophthalmic uses where ocular tissue is disinfected as part of the process of recovering and processing tissue for later use in transplant procedures and disinfecting surgical sites prior to surgical procedures. Those of skill in art will recognize, however, that the ultraviolet light delivery apparatus of the present invention can be adapted to other sanitization applications by varying factors such as the ultraviolet light wavelength, light intensity, exposure duration, or distance to a target surface.

The principals of operation for the ultraviolet light sanitization apparatus are discussed below before turning to the specific features of the apparatus embodiments disclosed in this application.

### Ultraviolet Light Sanitization

Sanitization effectiveness can be quantified in terms of the proportion of microbes that are eliminated following exposure to ultraviolet radiation. Microbes exposed to ultraviolet radiation show an exponential decrease in population as a function of exposure duration and increased radiation intensity. Sanitization effectiveness is commonly described with reference to a logarithmic decay function, such as: Ln(S(t))=K*I*t.

The variable “K” is a standard decay-rate constant that is unique to each type of microbe and that defines the sensitivity of the microbe to ultraviolet radiation. The variable “I” is the radiation intensity in microwatts per square centimeter and increases with increased power supplied to the light source and decreasing distance between the light source and the target surface. The variable “t” represents the duration of ultraviolet light exposure. The logarithmic decay function yields a normalized result where, for example, a 1-log exposure reduces microbe population by 90%, a 2-log exposure reduces microbe population by 99%, a 3-log exposure reduces microbe population by 99.9%, and each successive 1-log exposure results in a further order of magnitude reduction in the microbe population.

The consequence of the logarithmic decay with respect to the design and operation of an ultraviolet light delivery device is that the sanitization effectiveness will increase with decreasing distance between the light source and the target surface and increased ultraviolet light exposure duration. The ultraviolet light delivery apparatus of the present invention is designed to optimize sanitization effectiveness and consistency through control over the exposure duration times and the distance between the light source and target surface so that these factors are repeatable and consistent during use of the apparatus.

Sanitization effectiveness further depends on the wavelength of the ultraviolet light applied to the target surface. Ultraviolet light generally falls into one of three wavelength bands: (i) the UVA band having wavelengths between about 315 nm to 400 nm with minimal germicidal effect; (ii) the UVB band having wavelengths between about 280 nm to 315 nm with a moderate germicidal effect; and (iii) the UVC band having wavelengths between about 200 nm to 280 nm with the most significant germicidal effect of the three bands.

Ultraviolet light achieves the germicidal effect by being absorbed into proteins and nucleic acids of microbial cells and breaking down organic molecular bonds within microbial DNA resulting in cellular damage and death that inhibits microbial reproduction. This same DNA destroying effects observed in microbes is also observed in human and animal tissue and results in negative carcinogenic and cataractogenic effects.

To mitigate the negative carcinogenic and cataractogenic effects, the present ultraviolet light delivery apparatus is configured to emit narrow band UVC radiation with short wavelengths between 200 nm to 222 nm, which is referred to herein as “short UVC light.” In particular, the embodiments shown in the attached figures were constructed with an excimer flat lamp and filter design such that 90% of the emitted UV light falls at the 222 nm wavelength. Narrow band, short UVC light exhibits such a strong absorbance in biological materials that the short UVC light does not penetrate even the outer, non-living layers of skin or ocular tissue to reach the nuclei of living tissue cells. However, because microbes have comparatively smaller sizes, the short UVC light is still absorbed into the microbial DNA causing photonic disruption that damages the microbes. Thus, the short UVC light maintains effective sanitization while militating against the negative effects on human and animal tissue.

### Embodiments of the Disclosed Ultraviolet Light Delivery Apparatus

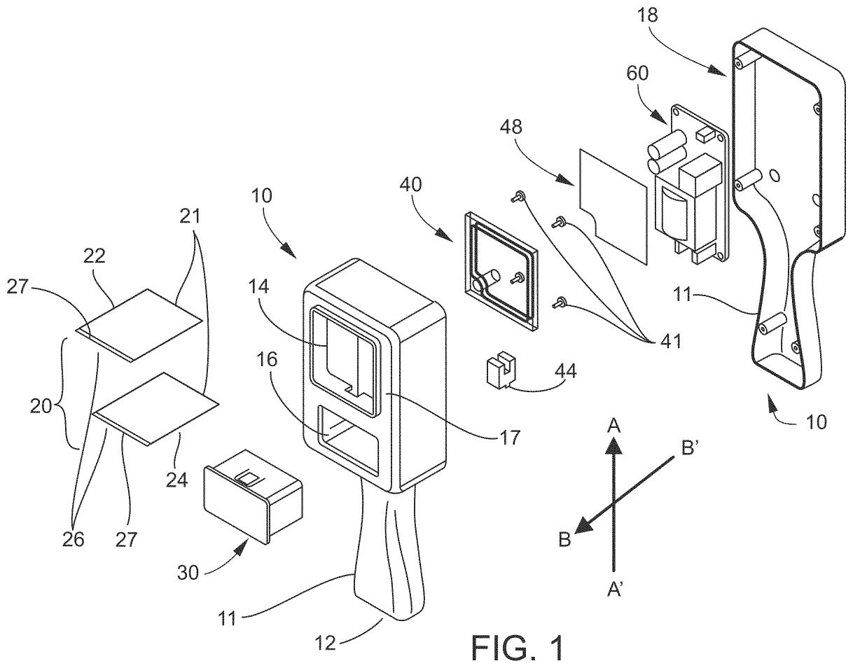

Turning to FIG. 1, a first embodiment of the ultraviolet light delivery device includes: (i) a case 10; (ii) a light source positioner 20; (iii) a timer relay 30; (iv) a light source 40; (v) a light source connector 44; (vi) a light filter 46 (not shown); (vii) light source fasteners 41; (viii) a light source reflective backing 48; and (ix) and a power supply 60. The case 10 includes a front portion 12 and a back portion 18 that can be secured together with threaded fasteners, snap fasteners, adhesive, or any other suitable securing mechanism known to one of skill in the art. The case front portion 12 includes a first cutout 14 and a second cutout 16 disposed on a front exterior surface 17 of the front portion 12 of the case 10. The first cutout 14 is sized to accommodate the light source 40 and a second cutout 16 sized to accommodate the timer relay 30. The case includes a handle portion 11 used by an operator to grip the apparatus during transport or use.

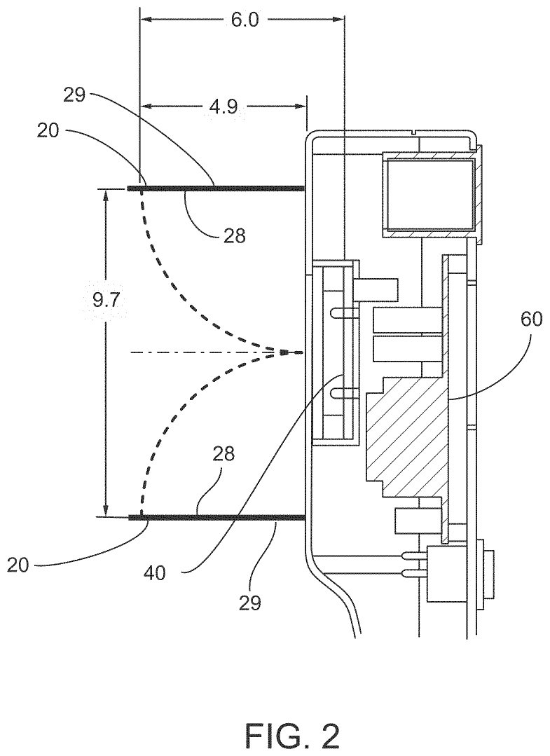

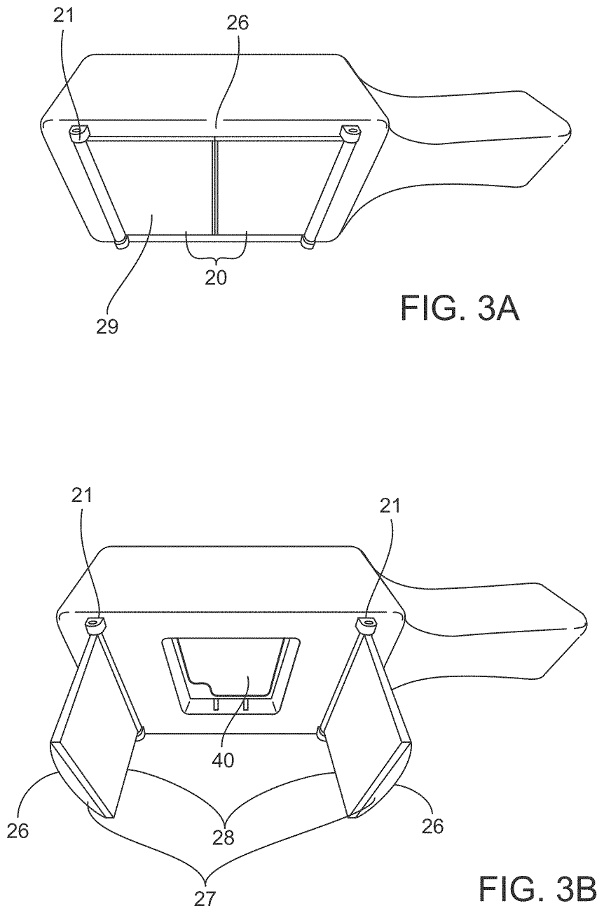

The light source positioner 20 shown in FIGS. 1, 2, and 3A-3B includes a first door 22 and a second door 24 that are rotatably secured to the case front portion 12 using hinges, for example. The first door 22 and the second door 24 can swing open to expose the light source 40 when the ultraviolet light delivery apparatus is in use and the light source 40 is emitting light. The doors 22 & 24 can swing closed to cover and protect the light source 40 when the ultraviolet light delivery apparatus is not in use.

The light source positioner 20 includes a proximal end 21 that can be releaseably, rotatably, or permanently secured to the case 10, and a distal end 26. The light source positioner 20 has a length, or operational distance, that spans between the proximal end 21 and the distal end 26. The operational distance or length of the light source positioner 20 can be sized to control the distance between the light source 40 and the target surface to be sanitized. For the light source positioner 20 shown in FIG. 1, the operational distance can be the same or can vary between the first door 22 and the second door 24. The light source positioner 20 further includes an outer surface 29 and an inner surface 28.

The light source positioner 20 can be made from, or covered with, a radiant shielding material or a radiant absorbent material that does not permit ultraviolet light to pass, which militates against the unnecessary spread of ultraviolet light to surrounding surfaces or tissue. For instance, the light source positioner 20 can be made from an ultraviolet light resistant material such as acrylics, high density polyethylene (“HDPE”), polycarbonate, or polyamides. The light source positioner 20 can include one or more ultraviolet light inhibitors, such as combinations of carbon black, rutile titanium oxide, hydroxybenzophenone, hydroxyphenylbenzotriazole, oxanilides for polyamides, benzotriazoles and hydroxyphenyltriazines for polycarbonate materials, or Hindered Amine Light Stabilizers (“HALS”), among other compounds known to those of skill in the art.

Alternatively, in other embodiments, the light source positioner 20 can be made from, covered, or coated with, a reflective material that likewise prohibits the unnecessary spread of ultraviolet light while also focusing the light on the target surface to be sanitized. Reflective materials can include, but are not limited to, thin metallic films of aluminum, lead, gold, silver, copper, or polymer materials such as polytetrafluoroethylene (“PTFE”) and polished acrylonitrile butadiene styrene (“ABS”). The reflective material can be coated on the light source positioner 20 to entirely or partially cover the inner 28 and/or outer surface 29.

The distal edges 26 of the first door 22 and the second door 24 shown in FIG. 1 include an approximately planar stabilizing surface 27 that can support the ultraviolet light delivery apparatus in a horizontal position when delivering light to a target surface (i.e., the A-A′ axis of FIG. 1 is parallel to the target surface). When the ultraviolet light delivery apparatus is supported on the stabilizing surfaces 27, the light source 40 is maintained a constant operational distance from the target surface while emitting ultraviolet light. This in turn facilitates delivery of a consistent intensity of ultraviolet radiation to the target surface during a single treatment and across multiple treatments, thereby ensuring that the antimicrobial, sanitizing effects remain consistent while also mitigating against the unnecessary spread of ultraviolet light that occurs when users position the apparatus too far from a target surface.

The first door 22 and second door 24 shown in the attached figures have an operational distance of approximately 5 cm long, and the light source 40 is installed within the case 10 approximately 1 cm from the front portion 12. Thus, the light source 40 maintains a consistent distance of approximately 6 cm from the target surface. This distance can be selected not only to optimize the sanitizing effects of the ultraviolet light delivery apparatus, but also to accommodate variations in the geometry of the target surface that may need to fit underneath the ultraviolet light delivery apparatus.

In some embodiments, the light source positioner 20 can be designed so it is easily removed from the case 10 and replaced with a light source positioner 20 of a different length to allow users to vary the intensity of ultraviolet light delivered to a target surface for different sanitizing applications. In yet other embodiments, the light source positioner 20 is configured so that the operational distance or length is extendible and retractable to again permit users to vary the intensity of ultraviolet light delivered to a target surface. Rather than being attached to the case with a hinge, the light source positioner 20 can be secured to the case 10 in a closed position with snap fit fasteners that allow the light source positioner 20 to be removed and re-secured to the case 10 in an open position. Other variations include use of a single door 22 that is designed to ensure appropriate spacing from a target surface. In the instance of a single door, a user can grip the handle portion 11 to stabilize the device during use, or the door 22 can incorporate a sufficiently large stabilizing surface 27 to balance the device during UV light operation.

In addition to the length of the light source positioner 20, the material and geometry of the light source positioner 20 can be varied to facilitate focusing the ultraviolet light on an intended target surface area. For instance, the light source positioner 20 can be made from a reflective material with a wider or curved geometry that helps focus the emitted ultraviolet light on a smaller target surface area.

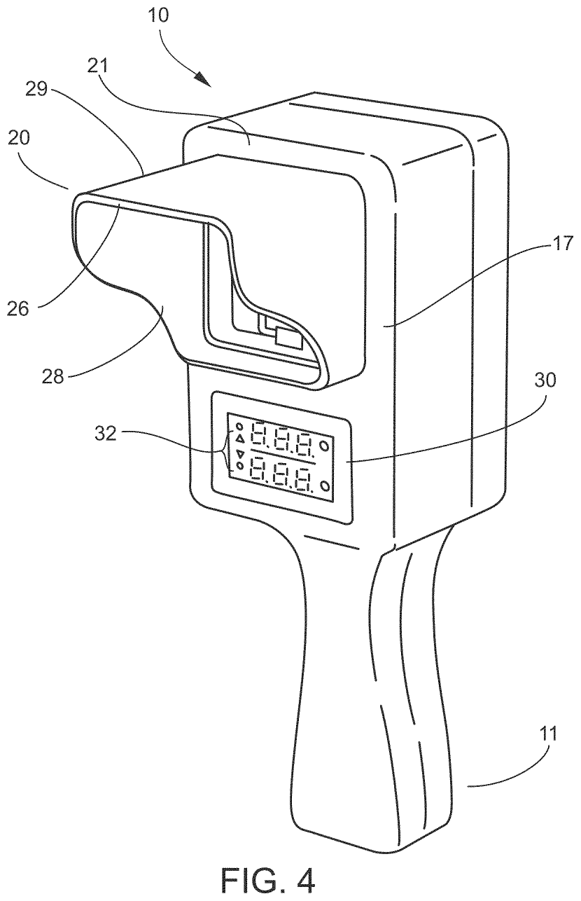

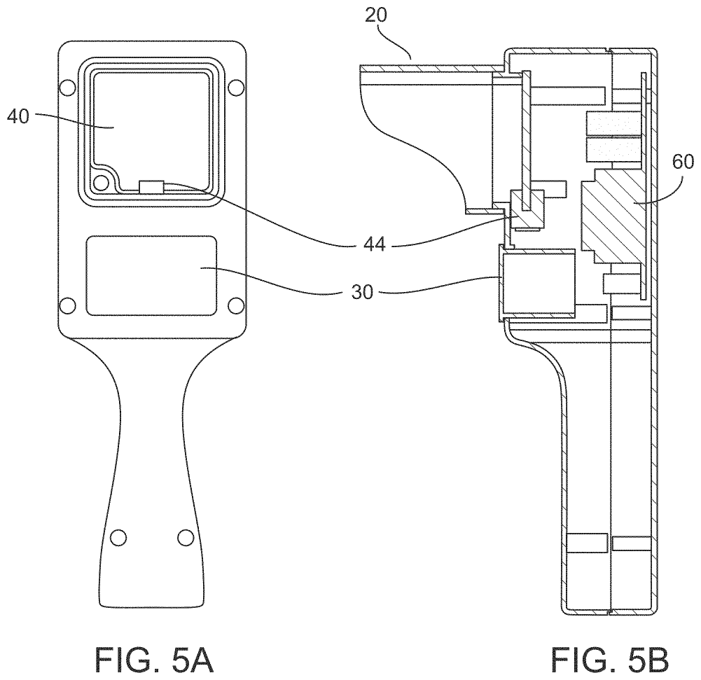

With reference to the embodiment shown in FIGS. 4 and 5A-5B, the light source positioner 20 is configured as a hood fixed to the case front portion 12 instead of the dual door design shown in FIGS. 1, 2, and 3A-3B. The hood can likewise be fabricated from radiant shielding or reflective material to prohibit unintended spread of ultraviolet light. In embodiments where the hood is constructed from a reflective material, the geometry of the hood can be varied to help focus the emitted ultraviolet light on a target surface.

The ultraviolet light delivery apparatus also includes an electronic timer relay 30 to control the dose of ultraviolet light delivered to a target surface and permit a further degree of control over the sanitizing effects of the apparatus. The timer relay 30 is placed in signal communication with the power supply 60 to the light source 40 such that power is supplied to the light source 40 when a timer countdown starts, and power is cut off from the light source 40 after a specified exposure duration period has elapsed. The electronic timer relay 30 can be an off-delay type timer with a programmable shut off delay corresponding to the desired exposure duration period.

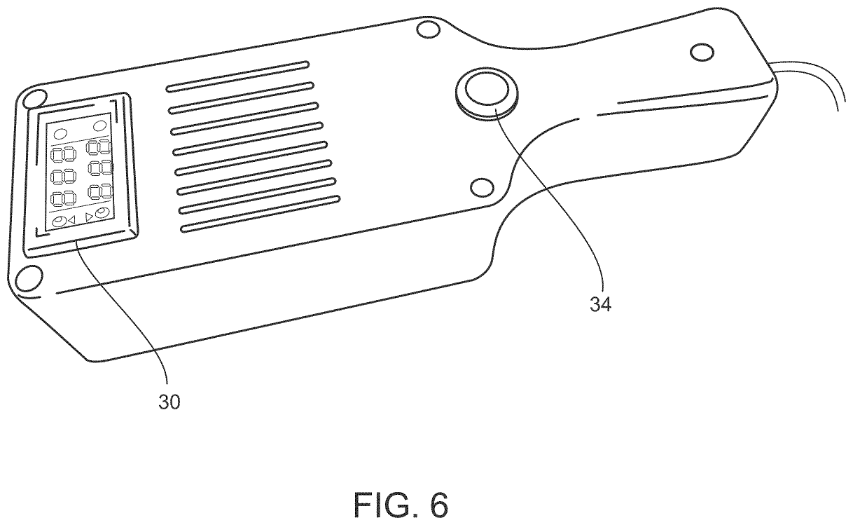

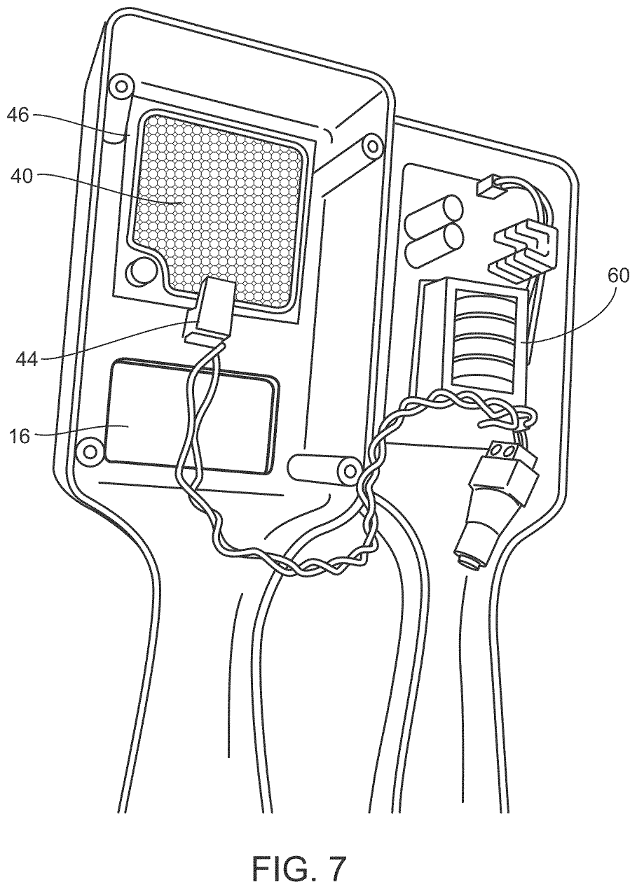

The timer relay 30 includes a digital display as well as one or more inputs (i.e., buttons, switches, etc.). The inputs include, for example: (i) one or more set point buttons 32, such as the up or down arrows shown in FIGS. 4 and 6, that permit users to specify an ultraviolet light exposure duration period (e.g., 30 seconds, 60 seconds, etc.); (ii) a start/stop button 34 to activate the timer and the light source 40 that can also be used to deactivate the light source 40 if light emissions are to be ceased before the timer expires; and (iii) a select button that allows users to toggle between displaying the timer countdown and a “total usage duration” for the light source 40.

With regard to the “total usage duration,” the timer relay 30 is connected to a digital storage device that permits the ultraviolet light delivery apparatus to calculate and store the total amount of time that the light source 40 has been in an “on” state emitting ultraviolet light. The total usage duration is output to the digital display of the timer relay 30. Tracking the total usage duration is an advantageous feature because light sources generally have a life span such that the intensity or even the wavelengths of the emitted light change over time with continued use. These changes can in turn negatively impact the sanitization efficacy and safety of an ultraviolet light source. After the total usage duration of a light source reaches a predetermined life span, the light source can be replaced to ensure continued consistency and efficacy in the delivery of ultraviolet light and sanitization. The timer relay 30 can then be reset to once again track the total usage duration of the replacement light source.

The example light source 40 shown in the attached figures, is an excimer flat lamp configured to emit ultraviolet light with a peak at 222 nm. The flat lamp has surface area dimensions of 50 mm×50 mm with a thickness of 1 mm. The light source 40 is secured to the case 10 using the light source fasteners 41 or any other suitable securing means that holds the light source 40 stable. The light source 40 is connected to the power source 60 through the light source connector 44.

The excimer flat lamp is constructed as a gas filled chamber that emits radiation with a wavelength that varies depending on the type of gas. The gas can include elements such as neon, argon, krypton, xenon, chlorine, fluoride, or bromide, among others. The elements combine in an excited state to form short-lived, high-energy molecular pairs called excited dimers or “excimers.” Radiation is emitted as the excimers disassociate, and excimers that emit radiation in the near UVC range can include Xe(2) (172 nm), ArCl (175 nm), KrI (190 nm), ArF (193 nm), KrBr (207 nm), and KrCl (222 nm). Various methods are used to place the gas in an excited state, including an electronic discharge, capacitive discharge, or dielectric barrier discharge that rely on two electrodes placed in contact with the gas chamber. The light source connector 44 shown in the attached figures can be configured as two electrodes that emit an electric current or as capacitive plates that produce a capacitive discharge.

The excimer flat lamp light source 40 emits ultraviolet light from both the front and rear surfaces (i.e., in both directions along the B-B′ axis of FIG. 1). Thus, the excimer flat lamp light source 40 has a front emission surface and a rear emission surface. The reflective backing 48 shown in FIG. 1 directs light emitted from the rear surface in the B′ direction back toward the B direction out of the first cutout 14 toward the target surface to be sanitized. The reflective backing 48 can be constructed from polished aluminum, thin film metallic materials, reflective polytetrafluoroethylene, or another suitable reflective material.

The ultraviolet light delivery apparatus can also include a filter 46 disposed between the light source 40 and the first cutout 14 where the filter 46 is configured to pass particular wavelength(s) of ultraviolet light. In one embodiment, the filter 46 passes ultraviolet light at the 222 nm wavelength so that 90% of the emitted light is at the 222 nm wavelength. This promotes safety by limiting the emission of higher wavelength ultraviolet light that carries carcinogenic and cataractogenic effects. The filter 46 can be fabricated from a fused silica material or a non-ultraviolet light inhibiting polymer material.

The power supplied to the light source 40 is preferably a consistent direct current (DC) supply so that the intensity of the emitted light and sanitizing effects do not vary over time. By way of example, the 222 nm excimer lamp shown in the attached figures utilizes a 12 volt DC input with a current of 900 milliamps (mA) that results in a lamp output of 6 kilovolts (kV) peek voltage. The required power can be supplied by rechargeable batteries so that the ultraviolet light delivery apparatus remains portable. Over an exposure duration of 60 seconds, the excimer lamp delivers approximately 12 milijoules per square centimeter (mJ/cm²) of energy at a distance of 5 cm from the lamp, which is below the maximum exposure guideline of 23 mJ/cm² published by the International Commission on Non-Ionizing Radiation Protection.

In some embodiments, the power supply 60 and light source 40 may generate excess heat that should be removed from the ultraviolet light delivery apparatus using heat sink fins or fans. To ensure proper operation the ultraviolet light delivery apparatus may need to stay below a specified operating temperature, such as 70 degrees Celsius depending on the materials utilized in the apparatus construction.

### Experimental Results

As part of the ultraviolet light delivery apparatus design, the inhibition of microbe growth was tested for varying durations of ultraviolet light exposure at varying distances between the ultraviolet light source and the target surface to be sanitized. In particular, the target surfaces were plates, or Petri dishes, deposited with concentrations of: (i) Candida Albicans (“CA”), a pathogenic yeast or fungus; (ii) Methicillin-resistant Staphylococcus aureus (“MRSA”), an infectious bacteria; and (iii) Candida Tropicana (“CT”), another pathogenic yeast or fungus.

The Petri dishes were exposed to a narrow band ultraviolet light source at distances of 2 mm, 4 mm, 5 mm, 5.5 mm, and 6 mm and at exposure durations of 5, 10, 20, 30, 40, and 60 seconds. The microbes were allowed to propagate, and microbe inhibition was observed at 24 hours and 48 hours after ultraviolet light exposure.

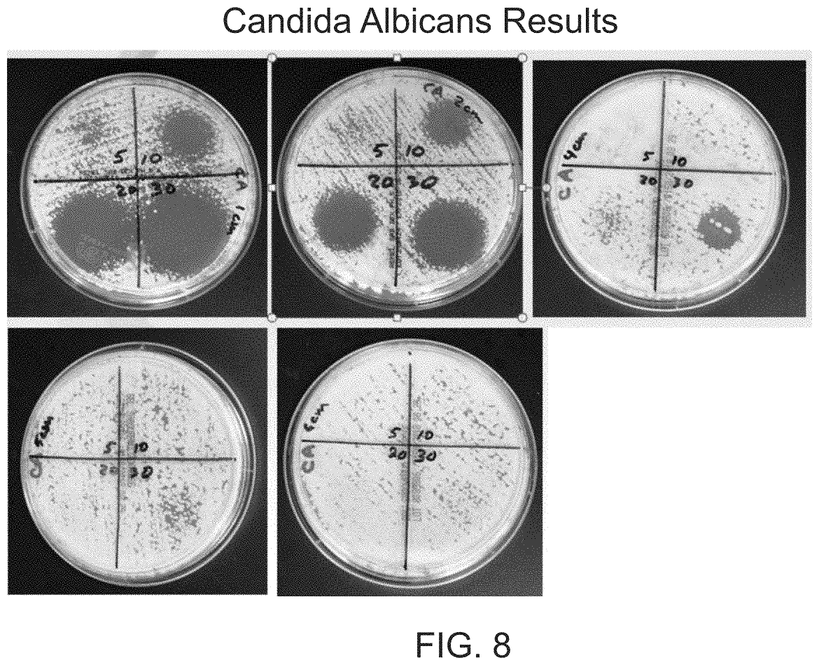

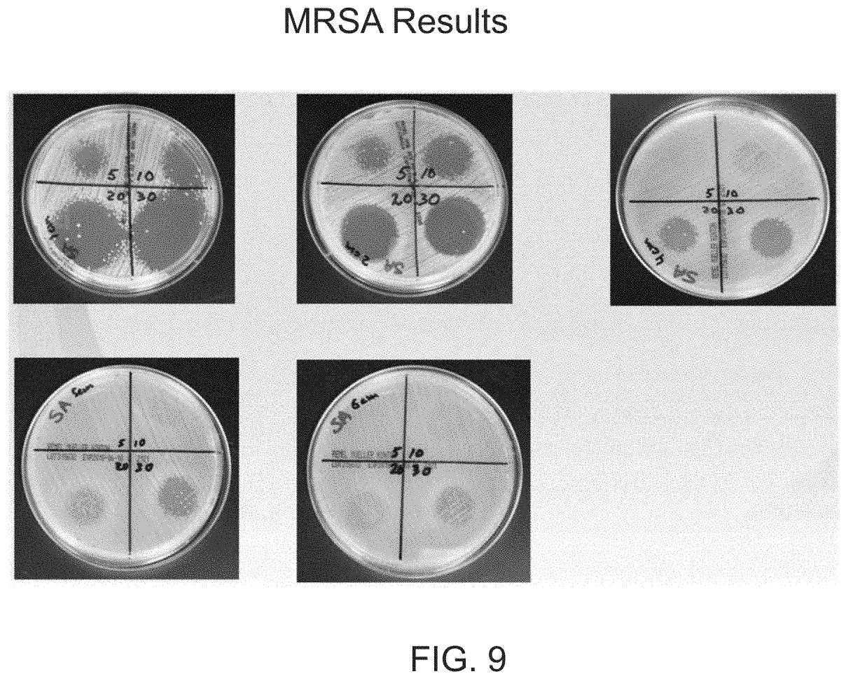

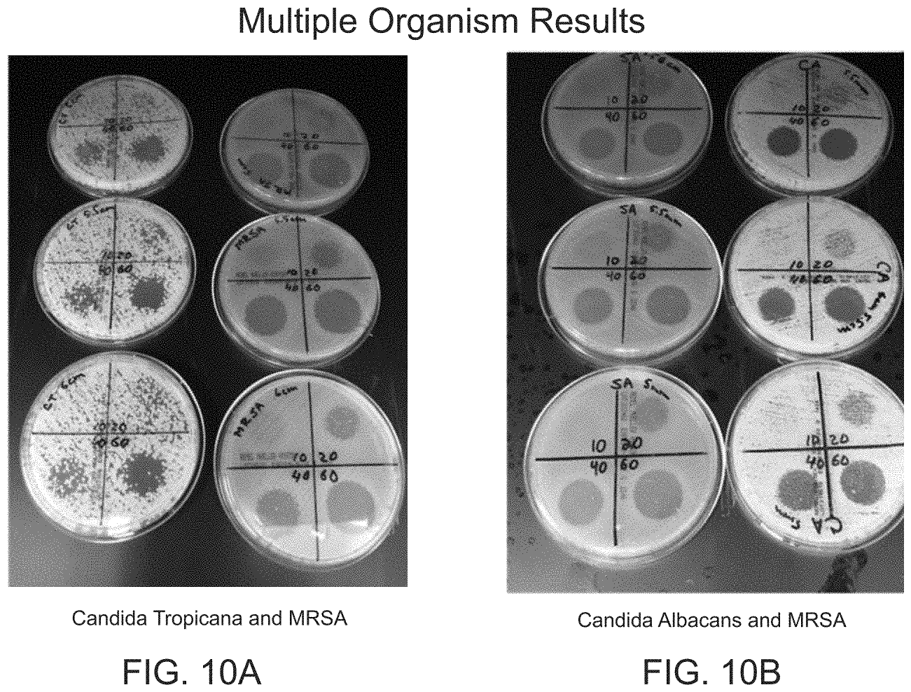

The results are shown in FIGS. 8, 9, and 10A-10B where the irradiated areas are shown as clear where microbe growth was inhibited and as clouded in other areas were microbe growth persisted. The results generally illustrate that microbe growth was inhibited with longer ultraviolet light exposure times and shorter distances between the target surfaces and the ultraviolet light source.

Distances of approximately 5 mm to 6 mm exhibited significant microbe growth inhibition but required longer exposure times of 40 to 60 seconds to achieve microbe growth inhibition. Thus, with a light source positioner 20 door 22 & 24 length of about 5 mm to 6 mm, the timer relay 30 should be configured to permit longer exposure durations of at least 30 to 60 seconds with the 222 nm excimer flat lamp light source.

Although the foregoing description provides embodiments of the invention by way of example, it is envisioned that other embodiments may perform similar functions and/or achieve similar results. Any and all such equivalent embodiments and examples are within the scope of the present invention.

What is claimed is:

1. An ultraviolet light delivery apparatus comprising:
(a) a case having
i. a front side with a front exterior surface,
ii. a rear side, and
iii. a first axis extending in a direction between the front exterior surface and the rear side;
(b) a light source positioner that comprises
i. a proximal end secured to the case, and
ii. a distal end that extends an operational distance outward from the front exterior surface along the first axis;
(c) an ultraviolet light source secured to the case, wherein the ultraviolet light source comprises a first emission surface that emits ultraviolet light in a direction along the first axis outward from the front exterior surface; and
(d) a timer relay secured to the case, wherein
i. the timer relay is in signal communication with a power supply, and
ii. the timer relay is configured to remove power to the ultraviolet light source after a specified exposure duration time period has elapsed.

2. The ultraviolet light delivery apparatus of claim 1, wherein the ultraviolet light source is an excimer lamp that emits ultraviolet light having at least one wavelength from 200 nanometers to 280 nanometers.

3. The ultraviolet light delivery apparatus of claim 1, wherein the ultraviolet light source is an excimer lamp that emits ultraviolet light having a wavelength of 222 nanometers.

4. The ultraviolet light delivery apparatus of claim 1, wherein:
(a) the ultraviolet light source further comprises a second emission surface that emits ultraviolet light in a direction along the first axis toward the rear side of the case; and
(b) the ultraviolet light delivery apparatus further comprises a reflective backing secured within the case and positioned between the ultraviolet light source second emission surface and the rear side of the case.

5. The ultraviolet light delivery apparatus of claim 1, wherein the ultraviolet light delivery apparatus further comprises an ultraviolet light filter positioned proximal to the ultraviolet light source first emission surface.

6. The ultraviolet light delivery apparatus of claim 1, wherein:
(a) the light source positioner distal end comprises a stabilizing surface that frictionally engages a target surface transverse to the first axis; and
(b) the target surface receives ultraviolet light emitted by the first emission surface.

7. The ultraviolet light delivery apparatus of claim 1, wherein a light source positioner inner surface is at least partially covered by an ultraviolet light reflective material.

8. The ultraviolet light delivery apparatus of claim 1, wherein a light source positioner inner surface is at least partially covered by a radiant shielding material.

9. The ultraviolet light delivery apparatus of claim 1, wherein the operational distance is four centimeters to seven centimeters.

10. The ultraviolet light delivery apparatus of claim 1, wherein the light source positioner is releasably secured to the front exterior surface by a hinge.

11. The ultraviolet light delivery apparatus of claim 1, further comprising a set point input control that interfaces with the timer relay to increase or decrease the specified exposure duration time period.

12. The ultraviolet light delivery apparatus of claim 1 further comprising:
(a) a digital processor in signal communication with the timer relay, wherein the digital processor is configured to determine a total usage duration for the ultraviolet light source based on data received from the timer relay; and
(b) a non-transitory electronic data storage device configured to store the total usage duration.

13. An ultraviolet light delivery apparatus comprising:
(a) a case having
i. a front side with a front exterior surface,
ii. a rear side, and
iii. a first axis extending in a direction between the front exterior surface and the rear side;
(b) an ultraviolet light source secured to the case, wherein the ultraviolet light source comprises a first emission surface that emits ultraviolet light in a direction along the first axis outward from the front exterior surface; and
(c) a light source positioner that comprises
i. a first door and a second door that each have a proximal end rotatably secured to the case and that each have a distal end located an operational distance from the proximal end,
ii. an open position wherein the first door distal end and the second door distal end both extend in a direction along the first axis outward from the front exterior surface, and
iii. a closed position wherein the first door distal end and the second door distal end are both adjacent to the front exterior surface, and the first door and the second door at least partially obscure the ultraviolet light source first emission surface.

14. The ultraviolet light delivery apparatus of claim 13, further comprising a timer relay secured to the case, wherein (i) the timer relay is in signal communication with a power supply, and (ii) the timer relay is configured to remove power to the ultraviolet light source after a specified exposure duration time period has elapsed.

15. The ultraviolet light delivery apparatus of claim 14, wherein the ultraviolet light source emits ultraviolet light having a wavelength of 222 nanometers.

16. The ultraviolet light delivery apparatus of claim 13, wherein:
(a) the ultraviolet light source is an excimer lamp that emits ultraviolet light having a wavelength of 222 nanometers;
(b) the ultraviolet light source further comprises a second emission surface that emits ultraviolet light in a direction along the first axis toward the rear side of the case; and
(c) the ultraviolet light delivery apparatus further comprises a reflective backing secured within the case and positioned between the ultraviolet light source second emission surface and the rear side of the case.

17. The ultraviolet light delivery apparatus of claim 13, wherein:
(a) the first door distal end and the second door distal end are both comprise a stabilizing surface that frictionally engages a target surface transverse to the first axis; and
(b) the target surface receives ultraviolet light emitted by the first emission surface.

18. An ultraviolet light delivery apparatus comprising:
(a) a case that comprises
i. a front side with a front exterior surface,
ii. a first cutout disposed on the front exterior surface,
iii. a second cutout disposed on the front exterior surface,
iv. a rear side,
v. a handle portion, and
vi. a first axis extending in a direction between the front exterior surface and the rear side;
(b) a light source positioner secured to the case proximal to the first cutout, wherein
i. the light source positioner comprises a first door with a first distal end and a second door with a second distal end,
ii. the first distal end and the second distal end each comprise a stabilizing surface that frictionally engages a target surface positioned transverse to the first axis, and
iii. the first door and the second door are secured to the case by a hinge;
(c) an ultraviolet light source secured to the case at least partially within the first cutout, wherein the ultraviolet light source comprises a first emission surface that emits ultraviolet light in a direction along the first axis outward from the front exterior surface;
(d) a timer relay secured to the case at least partially within the second cutout, wherein
i. the timer relay is in signal communication with a power supply, and
ii. the timer relay is configured to remove power to the ultraviolet light source after a specified exposure duration time period has elapsed.

19. The ultraviolet light delivery apparatus of claim 18, wherein:
(a) the ultraviolet light source is an excimer lamp that emits ultraviolet light having a wavelength of 222 nanometers;
(b) the ultraviolet light source further comprises a second emission surface that emits ultraviolet light in a direction along the first axis toward the rear side of the case; and
(c) the ultraviolet light delivery apparatus further comprises a reflective backing secured within the case and positioned between the ultraviolet light source second emission surface and the rear side of the case.

20. The ultraviolet light delivery apparatus of claim 19, wherein the ultraviolet light delivery apparatus further comprises an ultraviolet light filter positioned proximal to the ultraviolet light source first emission surface.
